SEQUENCE LISTING (2 sequences)

SEQ ID NO: 1 - DNA, 21 bp, Artificial Sequence
  primer
gccggatcaagagctaccaac

SEQ ID NO: 2 - DNA, 22 bp, Artificial Sequence
  primer
ggttgtaaaacgacggccagtg

(19) United States
(12) Patent Application Publication
(10) Pub. No.: US 2003/0162199 A1
(43) Pub. Date: Aug. 28, 2003

(54) Reversible chemical modification of nucleic acids and improved method for nucleic acid hybridization

(75) Inventor: Alex G. Bonner, Lexington, MA (US)

Correspondence Address: LAHIVE & COCKFIELD, 28 STATE STREET, BOSTON, MA, 02109, US

(73) Assignee: BioLink Partners, Inc., Watertown, MA

(21) Appl. No.: 10/264,295

(22) Filed: Oct. 3, 2002

Related U.S. Application Data
(63) Continuation of application No. PCT/US01/10901, filed on Apr. 3, 2001.
(60) Provisional application No. 60/194,288, filed on Apr. 3, 2000.

(51) Int. Cl. C12Q 1/68; C12P 19/34

(52) U.S. Cl. 435/6; 435/91.2

(57) ABSTRACT

The present invention relates to improved alternatives for hot-start PCR hybridization and amplification methods by using, in an embodiment, a heat-reversible, covalent modification of nucleic acids to disrupt hybridization of primer to template, or to interfere with the ability for the polymerase enzyme to recognize nucleoside triphosphates. In an illustrative embodiment, the amino groups of guanosine have been reversibly modified by reaction with glyoxal under mild conditions.

### RELATED INFORMATION

The present application is continuation of PCT patent application number PCT/US01/10901, filed on Apr. 3, 2001, which claims priority to U.S. provisional patent application serial No. 60/194,288, filed on Apr. 3, 2000, the entire contents of which are hereby incorporated by reference.

### BACKGROUND OF THE INVENTION

The copying and cloning of virtually any nucleic acid sequence has been greatly facilitated by the process of the polymerase chain reaction (PCR). Accordingly, the PCR has become a fundamental methodology in molecular biology.

In brief, the PCR involves hybridizing primers to the denatured strands of a target nucleic acid or template in the presence of a polymerase enzyme and nucleotides under appropriate reaction conditions. The polymerase enzyme (usually a thermostable DNA polymerase) then recognizes the primer hybridized to the template and processes a primer extension product complementary to the template. The resultant template and primer extension product can then be subjected to further rounds of subsequent denaturation, primer hybridization, and extension as many times as desired in order to increase (or amplify) the amount of nucleic acid which has the same sequence as the target nucleic acid. The details of the PCR are described in, e.g., U.S. Pat. Nos. 4,683,195 and 4,965,188 (Mullis et al.). Commercial vendors, such as Perkin Elmer (Norwalk, Conn.) market PCR reagents and publish PCR protocols.

The optimal efficiency of the amplification reaction, however, may be compromised by a number of unwanted side reactions. For example, many PCR procedures yield non-specific by-products caused by mispriming of the primers and template especially at lower temperatures when the reaction components are combined or during the first, denaturation step in the process when the temperature is brought from room temperature (“RT”) to 70-95° C. Primers hybridizing to each other may also result in lost efficiency. This problem may be particularly acute when the target nucleic acid is present in very low concentrations and may obscure any amplified target nucleic acid (i.e., may produce high background).

Several technologies have attempted to improve upon the standard PCR conditions by, e.g., encapsulating certain reagents (see, e.g., U.S. Pat. No. 5,643,764; Kosak, et al.), chemically modifying the polymerase (see, e.g., U.S. Pat. No. 5,773,258; Birch et al. and U.S. Pat. No. 5,677,152; Birch et al.), or inhibiting the polymerase with an antibody (see, e.g., U.S. Pat. No. 5,587,287; Scalice et al.) such that various components of the PCR are released for reaction only at the proper time. There are, however, limitations to each of these methods.

For example, not all PCR reagents are conveniently encapsulated or efficiently released from the encapsulation substrate. Moreover, chemical modification of the polymerase frequently compromises full enzymatic activity. Antibodies that inhibit the polymerase are unlikely to have any direct effect on undesired nucleic acid interactions (e.g., primer/template mispriming or primer/primer hybridization).

The PCR and other important genomic methods, such as SNP (single nucleotide polymorphism) analysis and allele-specific oligonucleotide (ASO) hybridization which are the basis for microarray or DNA-Chip methods, and DNA sequencing, leverage the property of nucleic acids to hybridize, and all these methods share the same problem of unwanted hybridization.

Hot-Start PCR has been described as “a common and easy protocol to improve yield and increase specificity.” During sample preparation at room temperature complexes of nonspecific primer-template may be generated. With the Hot Start method a key component necessary for amplification, such as primers, polymerase, Mg++, or dNTPs, is withheld from the reaction mix until the reaction reaches a temperature above the optimal annealing temperature of the primers. Competing side reactions are therefore minimized somewhat, but not eliminated.

### SUMMARY OF THE INVENTION

Improved methods of amplifying nucleic acid sequences and methods of inhibiting the formation of undesired amplification products are presented which, in an embodiment, are well suited for performing the PCR, preferably hot-start PCR, on a target nucleic acid. The methods employ reversibly chemically modified moieties in the amplification reaction mixture, which reduce or eliminate primer/template mispriming, or primer/primer hybridization. The reversibly chemically modified moieties include the target nucleic acid(s), primer(s) or nucleoside triphosphates in the reaction mixture. In a preferred embodiment, the chemical modification includes a removable protecting group which can then be conveniently released from the nucleic acids, e.g., those involved in Watson-Crick hydrogen bonding, using, e.g., heat. Hence, non-specific amplification products are eliminated or greatly reduced because amplification does not occur until after the initial incubation period at elevated temperature (which removes the reversible protecting group on the nucleic acids.)

Accordingly, in one aspect, the invention provides a method for selectively amplifying a target nucleic acid, including combining a target nucleic acid under conditions which allow for an amplification reaction to occur. The amplification reaction includes one or more target nucleic acids, primers or nucleoside triphosphates which have been reversibly modified so as to inhibit the formation of undesired amplification products, thereby forming a resultant mixture resulting in the selective amplification of the target nucleic acid.

In another embodiment, the polymerase is E. coli DNA polymerase I, Klenow fragment of E. coli DNA polymerase I, T4 DNA polymerase, reverse transcriptase, and preferably, thermostable DNA polymerase.

In another embodiment, the resultant reaction mixture is subjected to at least one thermal cycle.

In another embodiment, the nucleic acid is RNA and may be incubated, for example, with a reverse transcriptase, and then a DNA polymerase, such as a thermostable DNA polymerase.

In a second aspect, the invention provides an amplified nucleic acid produced by a process including combining a target nucleic acid under conditions which allow for an amplification reaction to occur. The amplification reaction includes one or more target nucleic acids, primers or nucleoside triphosphates which have been reversibly modified so as to inhibit the formation of undesired amplification products, thereby forming a resultant mixture resulting in the selective amplification of the target nucleic acid.

In one embodiment, the polymerase is a thermostable DNA polymerase, and preferably, the reaction mixture is subjected to at least one thermal cycle.

Another embodiment of the invention relates to a kit for conducting a polymerase amplification reaction, containing a reagent for reversibly chemically modifying a nucleic acid or nucleobase such that when the nucleic acid or nucleobase is used in a polymerase amplification reaction, the formation of undesired amplification products is inhibited; and instructions for use. The kit may desirably include a component such as a nucleic acid, a nucleic acid primer, a modified nucleic acid primer, a nucleotide, a modified nucleotide, and a nucleic acid polymerase, such as a thermostable DNA polymerase, and most desirably includes at least one other component for conducting a polymerase amplification reaction, such as a buffer or reagents suitable for modifying a nucleic acid.

In a further related embodiment, the kit includes a removable protecting group comprising glyoxal or glyoxal analogues or derivatives. Such glyoxal analogues and derivatives include glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal.

Other removable protecting groups in accordance with the invention include amides such as trifluoroacetyl, trichloroacetyl, which groups can be reversibly chemically attached to a nucleotide (or nucleoside) using, e.g., acetic, trifluoroacetic, or trichloroacetic anhydrides or, e.g., 3,4,5,6-tetrahydrophthalic anhydride (see, e.g., Gibbons et al. Biochem. v. 15 no. 1, 52-60 (1976)); γ-Carboxyacylamides such as acontinoyl, maleyl, citriconyl, phenoxyacetyl, or acetoacetyl, which groups can be reversibly chemically attached to a nucleotide using maleic, citriconic, 2,3 methyl maleic and tetrafluorosuccinic anhydride, phenoxyacetic acid, or diketene; amidines such as imidoamides, which groups can be reversibly chemically attached to a nucleotide using methyl acetimidate-HCL, methyl benzimidate-HCL; carbamates such as ethoxycarbonyl, which groups can be removably derivatized to a nucleotide using ethoxyformic anhydride; and others including N-silyl, imine, orthoester, etc. Other useful reagents for providing removable protecting groups include 1,2-dicarbonyl compounds including 3-ethoxy-2-ketobutyraldehyde(kethoxal), ninhydrin, hydroxyacetone, diethyl oxalate, diethyl mesoxalate, 1,2-naphthoquinone-4-sulfonic acid, and pyruvaldehyde.

Another aspect of the invention relates to a compound having the ability to amplify a target nucleic acid and reduce undesired amplification products, comprising a removable protecting group. This compound may desirably comprise a reaction mixture containing a removable protecting group and guanosine 5′-triphosphate.

In one embodiment of this invention, the removable protecting group is glyoxylaldehyde having the general formula:

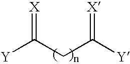

where n=0 to 4; Y and Y′ are selected from Cl, OH, H, NR₂, and OR; R is an alkyl group, e.g., methyl, ethyl or isopropyl; and X and X′ are independently O or S.

In a further related embodiment, the kit includes a removable protecting group comprising glyoxal or glyoxal analogues or derivatives. Such glyoxal analogues and derivatives include glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal. Other features and advantages of the invention will be apparent from the following detailed description and claims.

In an embodiment, the removable protecting group forms a heterocyclic group on the six-membered heterocyclic group of the nucleotide or nucleoside, e.g., guanine or adenine, thus preventing Watson-Crick hydrogen bonding. In another embodiment, the removable protecting group is added to the free amino group of the nucleoside.

The invention will be useful in commercial applications including modified nucleic acids, specialty chemicals, and instrumentation for utilizing this technology, e.g., probe based diagnostics, microarray/DNA Chip methods, PCR (including hot-start PCR) hybridization and amplification, SNP analysis, and DNA sequencing. Other applications include drug discovery and the study of drug response genes (pharmacogenomics), drug delivery and therapeutics.

An advantage of the invention is that the methods require no manipulation of the reaction mixture following initial preparation. Thus, the invention may be used in existing automated PCR amplification systems (including hot-start PCR hybridization and amplification) and with in situ amplification methods where the addition of reagents after the initial denaturation step is inconvenient or impractical. The invention may also be used for controlled release technology of nucleic acids in drug delivery of antisense DNA therapeutics, and anti-cancer drugs such as AZT and dideoxy nucleotides, and as potential intermediates to facilitate the delivery of DNA for non-viral transfection and gene therapy.

### BRIEF DESCRIPTION OF THE DRAWINGS

FIG. 1 is a graphical representation of DMT-isobutryl-Guanosine being converted to the DMT-Guanosine model compound by treatment with ammonium hydroxide (28%);

FIG. 2 is a graphical representation of 5′DMT-Guanosine being treated with glyoxal to obtain the Glyoxylated-5′DMT-Guanosine;

FIG. 3 is an overlay of reversed phase HPLC chromatograms. The lower trace is the DMT-Guanosine starting material; the middle trace is the Glyoxylated-DMT-Guanosine product, and the upper trace (offset by 1 min.) is a 1:1 mixture of starting material and product;

FIG. 4 is a time study for hydrolysis of Glyoxylated-DMT-Guanosine. A sample of Glyoxylated-DMT-Guanosine was allowed to stand at 55° C. in a sealed tube with tris-borate buffer pH 8.3. Aliquots were analyzed by reversed phase HPLC and the areas of starting material and product (DMT-Guanosine) were determined and plotted. The half life of the glyoxylated intermediate under these conditions is about 240 min. and the half-life at 95° C. is estimated to be 3-15 min. (a 16 to 81-fold rate increase caused by the temperature increase);

FIG. 5 is MALDI-TOF-MS data for the unmodified P-1 primer and the glyoxylated P-2 primer;

FIG. 6 shows comparison results of PCR amplification of pUC using unmodified primers (lane 2), glyoxylated primers (lane 3), glyoxylated primers with 10 min. preincubation at 95° C. (lane 4), and unmodified primers with 10 min. preincubation at 95° C. (lane 5). Identical amplification conditions including concentration of the pUC template and primers were used for this comparison. This is a result of an electrophoresis performed at 70 mA for 2-3 hours. The Gels were visualized using a Fisher Scientific Transilluminator and were imaged using a Kodak DC120 Digital Zoom Camera;

FIG. 7 shows pictorially reversible chemically modified groups identified as “X” or Compound-X blocking hybridization;

FIG. 8 shows amino groups of the four nucleobases as available nucleophilic sites which are directly involved in Watson-Crick hydrogen bonds;

FIG. 9 shows nucleic acids modified with readily available, small molecule protecting groups that can be easily and efficiently introduced to sites that disrupt complementarity and heteroduplex formation;

FIG. 10 shows hybridization of an oligonucleotide with a complementary strand of DNA, where a single base mismatch results in a thermal melting point (Tₘ) that is about 10° C. lower than the Tₘ of a perfectly complementary duplex; and

FIG. 11 shows a representative reaction scheme directed at nucleobase amino groups (e.g. N⁴ of cytidine, N⁶ of Adenine, N¹ or N² of Guanine, and N³ of Thymine) using a cyclic anhydride as the modifying group.

### DETAILED DESCRIPTION OF THE INVENTION

As used herein the term “nucleic acid” includes DNA molecules (e.g., cDNA or genomic DNA), RNA molecules (e.g., mRNA), analogs of the DNA or RNA generated using nucleotide analogs or using nucleic acid chemistry, and PNA (protein nucleic acids). In addition, the nucleic acid molecule can be single-stranded or double-stranded.

The term “target nucleic acid” or “template” includes any nucleic acid intended to be copied in, e.g., a polymerase amplification reaction such as the PCR.

The term “undesired amplification products” includes nucleic acid sequences other than the target sequences which result from primers hybridizing to sequences other than the target sequence and then serving as a substrate for primer extension. The hybridization of a primer to a non-target sequence is referred to as “non-specific side products.” This also can occur during the lower temperature, reduced stringency pre-reaction conditions.

The term “primer” or “nucleic acid primer” includes short single-stranded oligonucleotides that, typically, are between about 10 to 100 bases and are designed to hybridize with a corresponding template nucleic acid. Primer molecules may be complementary to either the sense or the anti-sense strand of a template nucleic acid and are typically used as complementary pairs that flank a nucleic acid region of interest.

The term “removable protecting group” includes the group which is reversibly chemically attached to the reversibly chemically modified moieties (including the target nucleic acid(s), primer(s) or nucleoside triphosphates in the reaction mixture), and alternately, where appropriate, may refer to the reagent, e.g., glyoxal, which reversibly chemically modifies the nucleotide or nucleoside.

The term “polymerase” includes any one of, or a mixture of, the nucleotide polymerizing enzymes E. coli DNA polymerase I, TAQ polymerase, Klenow fragment of E. Coli DNA polymerase I, T4 DNA polymerase, reverse transcriptase where the template is RNA and the extension product is DNA, or a thermostable DNA polymerase.

The term “thermostable DNA polymerase” includes a thermostable DNA polymerase isolated from Thermus aquaticus, Thermus thermophilus, Thermus filiformis, Thermus flavus, Pyrococcus furiosus, Thermococcus literolis, a Thermotoga species, or a recombinant form thereof.

The term “nucleotides” refers to any nucleotide (including modified nucleotides such as methylated or biotinylated nucleotides) that can be incorporated into a nucleic acid by a polymerase.

The term “selective amplification” refers to the preferential copying of a target or template nucleic acid of interest using a polymerase amplification reaction, such as the PCR.

The term “thermal cycle” includes any change in the incubation temperature of a nucleic acid sample designed to change the activity of a component of the sample such as, e.g., the binding affinity of a primer for a nucleic acid.

The terms “hybridize” or “hybridization” are art-known and include the hydrogen bonding of complementary DNA and/or RNA sequences to form a duplex molecule. Typically, hybridization takes place between a primer and template but may also take place between primers and these reactions, when undesired or unscheduled, can be inhibited by using methods and compositions of the invention.

The terms “amplification” or “amplify” include the reactions necessary to increase the number of copies of a nucleic acid sequence, such as a DNA sequence. For the purposes of the present disclosure, amplification refers to the in vitro exponential increase in copy number of a target nucleic acid sequence, such as that mediated by a polymerase amplification reaction such as the PCR. Other amplification reactions encompassed by the invention include RT-PCR (see, e.g., U.S. Pat. No. 4,683,202; Mullis et al.), and the ligase chain reaction (Barany, Proc. Natl. Acad. Sci. USA 88:189-193 (1991)).

A polymerase amplification reaction such as the PCR involves hybridizing primers to strands of the target nucleic acid (template) in the presence of a polymerizing enzyme and deoxyribonucleotide triphosphates (dNTPs). The result is the formation of primer extension products along the templates that are complementary to the template. Once the primer extension products are denatured, one copy of the template has been prepared and the cycle of priming, extending, and denaturation can be carried out many times to provide an exponential increase in the amount of target template, e.g., for detection or further manipulation (e.g., cloning).

Unfortunately, however, many amplification procedures yield non-specific side products of nucleic acids that have not been targeted. Non-specificity is caused by mispriming of the primers by annealing to non-targeted sites. In addition, PCR procedures can sometimes yield primer-dimers or oligomers and double stranded side products containing the sequences of several primer molecules joined end to end. All of these unwanted products adversely affect accurate and sensitive detection of the targeted nucleic acid.

Most mispriming and side product formation is prevalent in the pre-PCR phase of the procedure, or the so-called “ramp up” period when the reaction mix is initially being heated to the denaturation temperature. Formation of non-specific products prior to the first cycle allows for these non-specific products to be amplified together with the target sequence causing the amplification reaction to produce false bands and efficiency.

This invention provides reversible, covalent, chemical modifications that can be applied advantageously to “hot-start” PCR amplification and/or hybridization. In ‘hot-start’ PCR, at least one reaction component, which can include the polymerase, salt (e.g., KCl or MgCl₂) or dNTP(s), is withheld from the reaction until the system reaches a particular temperature. The critical ‘hot-start’ temperature varies according to the method of delayed reaction. Reactions of exocyclic substituents of nucleobases are known in the art, e.g., N. K. Kochetkov, et al., Organic Chemistry of Nucleic Acids, Part B, Plenum Press, London and New York, 1972. The chemical behavior of the amino groups in nucleic acids closely resembles that of aromatic amines containing strong electron-acceptor substituents. However, nucleosides contain other functional groups capable of reacting with electrophilic reagents, such as the nitrogen atoms of the heterocyclic ring and the hydroxyl groups of the monosaccharide residue. In moving from nucleosides to nucleotides, the phosphate residue adds a functional group with strong nucleophilic properties and more side reactions become possible. In polynucleotides, a targeted chemistry for the nucleobases is further complicated by the variety of functionalities presented in heteropolymers and deleterious side-reactions such as acid catalyzed depurination and/or hydrolysis of the ribophosphate backbone. Hence, reactions directed only at the substituents of the heterocyclic ring of the nucleobases in a polynucleotide are difficult.

Of the three principal nucleosides containing amino groups, cytidine is the most readily acylated. See, for example, N. K. Kochetkov, et al., above. Selective N-acylation of the amino group of cytidine in nucleotides and polynucleotides has been achieved with acetic anhydride. See, for example, A. M. Michelson, J. Chem. Soc., 3655 (1959). It has been shown to decrease the ability of homopolymers to induce incorporation of amino acids in a cell-free system of protein synthesis. See, for example, A. M. Michelson, et al., Biochim. Biophys. Acta 91:92 (1964). However, this reaction product is not easily reversible under the conditions of PCR and is of little value for hot-start applications. Other anhydrides have been applied for reversible chemical modification of amino groups in protein chemistry but they have not been applied to nucleotides.

In regard to reactions with aldehydes, the reduced vulnerability of the amino group of the heterocyclic bases of nucleic acids to electrophilic attack has the effect that these compounds can react only with the most active carbonyl compounds. The ability of formaldehyde to hydroxymethylate amino groups of the heterocyclic ring is well documented, but is of little value because of the instability of the primary reaction products and the slow formation of stable, not-easily-reversible, reaction products.

The reaction between nucleotides with aldehydes containing additional functional groups whose interaction with the heterocyclic ring can lead to stabilization of the reaction product is more promising. An example of this approach is the reaction between amino components of nucleic acids and the alpha-oxide of acrolein. This reagent reacts selectively with guanosine to form a stable tricyclic end product and the other usual nucleosides do not react. The reaction between guanosine and alpha-dicarbonyl compounds proceeds in a similar manner. The exocyclic amino group of guanine and the N1 nitrogen atom take part in the reaction such that a tricyclic end product is formed. The product formed by the reaction between guanosine and glyoxal or 3-ethoxybutanon-2-al-1 (ketoxal) is stable in acid medium, and, in alkaline medium, it decomposes rapidly with the regeneration of guanosine (half-conversion time at pH 10 at RT about 24 h). see, e.g., R. Shapiro et al., J. Biol. Chem. v.245 no. 10, 2633-2639 (1970), and R. Shapiro et al., Biochem. v.5 no. 9, 2799-2807 (1966)

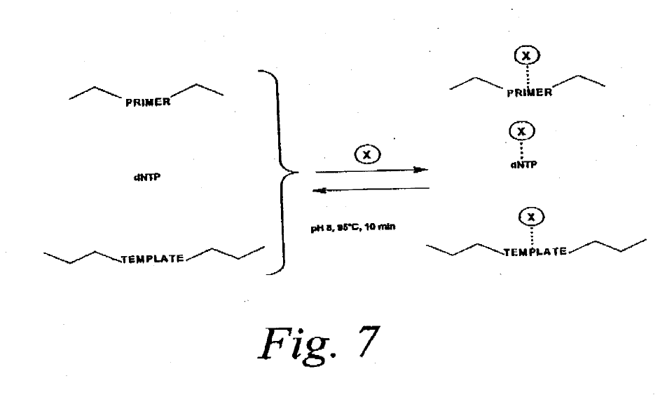

A preferred method of the invention performs the PCR using reversibly chemically modified nucleobases that are present in either the primer or the template. The presence of the reversibly chemically modified nucleobase(s) prevent the nucleic acid from hybridizing specifically or non-specifically. When the reaction mixture is heated, the removable protecting group is detached from the modified nucleic acid, and the primer and target molecules are able to hybridize. This is depicted in FIG. 7; the modifying groups are identified as “X” or Compound-X. As noted above, a heat release of the removable protecting group is preferred, but other methodologies include light-mediated inactivation, detergents, or physical disruption.

In another embodiment, the invention provides a method for detecting the presence or quantity of a target nucleic acid molecule that is present in low amounts or, for example, in the presence of background nucleic acid molecules.

Conditions, reagent concentrations, primer design, and appropriate apparatus for typical cyclic polymerase amplification reactions such as the PCR are known in the art. Moreover, there exist numerous variations on the typical amplification reaction which may be adapted to the methods of the invention. The PCR itself is described in, e.g., U.S. Pat. Nos. 4,683,195 and 4,683,202. Alternatively, a ligation chain reaction (LCR) may be used (see, e.g., Friedhoff, P. et al. (1988) Science 241:1077-1080; and Nakazawa et al. (1994) Proc. Nati. Acad. Sci. USA 91:360-364) or, e.g., RT-PCR (Mullis (1987) U.S. Pat. No. 4,683,202). The invention has the advantage of being conveniently incorporated into established protocols without the need for extensive re-optimization.

For detecting the resultant PCR product, any art recognized technique may be used, such as agarose gel electrophoresis, as described herein. Alternatively, the resultant products of the amplification reaction may be detected using a detectable label, that is, e.g., isotopic, fluorescent, calorimetric, or detectable e.g., using antibodies.

Accordingly, the amplification methods of the invention may be advantageously used to amplify virtually any target nucleic acid such as a nucleic acid fragment, gene fragment (e.g., an exon or intron fragment), cDNA, or chromosomal fragment.

The methods and compositions of the invention permit the detection and amplification of small amounts of nucleic acid and, as such, are applicable to a variety of uses including, diagnostic applications, research, and forensic science.

Reversible, covalent modification of nucleic acids according to the invention, using, e.g., glyoxal, to modify residues such as guanosine has a number of advantages. The conditions for the chemistry are extremely mild and can be applied to nucleic acids in aqueous conditions at pH levels where the nucleic acids are both soluble and stable. The reaction is fast and the reagent is not expensive. Also, excess glyoxal reagent can be removed by a variety of isolation methods appropriate for nucleic acids and the volatility of glyoxal allows for simple removal of excess reagent under vacuum conditions.

In the methods of the invention, incubation of the nucleic acids at a temperature which is equal to or higher than the primer hybridization (annealing) temperature used in amplification reactions insures amplification specificity by eliminating the non-specific chemistries that occur at lower temperatures. The length of incubation required to recover the activity of the nucleic acids depends on the temperature and pH of the reaction mixture and on the stability of the protecting group.

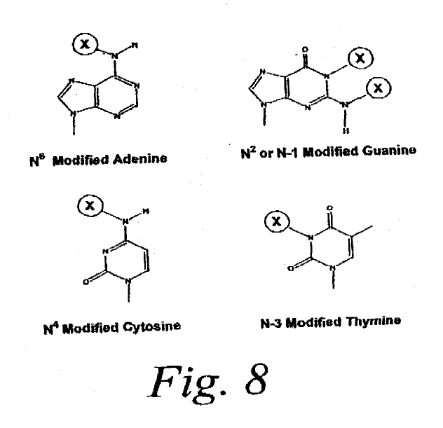

Nitrogen groups are desirable targets for a reversible, functional transformation. As shown in FIG. 8, amino groups of the four nucleobases are the most nucleophilic sites available and are directly involved in the Watson-Crick hydrogen bonds. Also, the carbonyl groups on G and T which exist as keto-enol tautomers are secondary candidates for modification.

Optimization of conditions for (a) reactivation of nucleic acids and (b) for reaction mixtures and concentrations may be determined by routine experimentation. In general, a pre-incubation is carried out in the amplification reaction buffer at a temperature greater than about 50° C. for between about 10 seconds and about 20 minutes. Typically, PCR amplification is performed using a thermostable DNA polymerase with reversibly modified primers. The annealing temperature used in the PCR amplification is 55° C.-75° C., and the pre-reaction incubation is carried out at a temperature equal to or higher than the annealing temperature, preferably a temperature greater than about 90° C. The amplification reaction mixture is usually incubated at 90-95° C. for 12-15 minutes to regenerate the unmodified nucleic acids prior to the temperature cycling of the PCR protocol. Suitable pre-reaction incubation conditions typically depend on the concentration of the nucleic acids (primers, template, or nucleoside triphosphates) used in the reaction and is relative to the rate of reaction for the removal of the glyoxal protecting group. Thus, optimum pre-reaction incubation conditions are determined experimentally and tailored to concentrations and reaction conditions being used for the specific amplicon of interest and the specific PCR protocol chosen for the experiment.

In a preferred embodiment of the invention, any of the foregoing kits may be further designed, packaged, or provided with instructions such that the kit may be conveniently used with a polymerase, such as a thermostable DNA polymerase, e.g., Taq available from Sigma, AmpliTaq available from ABI, or Pfu available from Life Technologies.

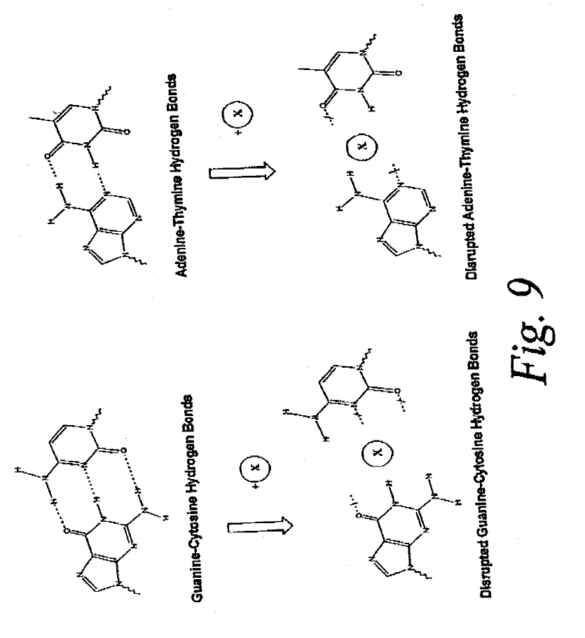

Nucleic acids modified with readily available, small molecule protecting groups that can be easily and efficiently introduced to sites that disrupt complementarity and heteroduplex formation as shown in FIG. 9.

The modifying group or groups are desirably stable to reaction and work-up conditions appropriate for nucleic acids, and to techniques for separation and purification such as chromatography, ethanol precipitation, etc. By design, the modified nucleic acids are stable but unable to hybridize with other nucleic acids in solution at room temperature. Ideally, deprotection of the modified nucleic acids results from simply increasing the temperature of the solution and behave according to the Arrhenius equation which relates temperature with reaction rate and corresponds to the common statement that a reaction rate increases by a factor of 2 to 3 for each 10° C. rise in temperature Thus, for instance, when the reaction temperature is raised from 25° to 95° C., a deprotection reaction rate increase of 2⁷ to 3⁷ (128 to 2187-fold) can be expected. Hence, deprotection occurs after a transient incubation at an elevated temperature, making the natural nucleic acid available to the solution and to the genomic method of choice.

While an ideal modifying group is desirably one that reacts reversibly at all bases (i.e. at A, C, G and/or T residues) or at multiple sites with similar reactivity, i.e. nucleophilicity, a selective chemistry for a single site at one nucleobase will be sufficient to reversibly disrupt hybridization in most cases. For instance, if the N⁴-amino group of all cytosine residues could be universally modified, then it is expected that this chemistry would be sufficient to prevent heteroduplex formation. Thus, a specific chemistry should be appropriate for all cases except those where cytidine or deoxycytidine are not included in the structure. This is not expected to be a limitation since most oligonucleotide primers consist of 15-20 residues with at least one or two residues of each nucleobase. For genomic methods that require dNTP's or NTP's, it is expected that reversible blocking of a single nucleoside triphosphate may be sufficient to cause a reversible disruption of the process. And, there should be no limitation of a site specific chemistry applied to genomic DNA where all four nucleobases are typically available.

A site specific chemistry with less than quantitative yield applied to an oligonucleotide with multiple reactive sites is expected to be sufficient to block hybridization in the majority of applications. For example, an oligonucleotide with only two cytidines and a 90% modification efficiency per site would have a mixture of protected products (81% at both sites, 18% at one site) and only 1% of the unmodified oligomer. If a site specific chemistry is applied to genomic DNA with even a 50% yield, then the sample should be denatured, essentially single stranded, and unable to participate in non-specific reactions or hybridizations. For genomic applications involving modified nucleoside triphosphates, a purified and well characterized product is preferred and will be obtained after appropriate purification steps.

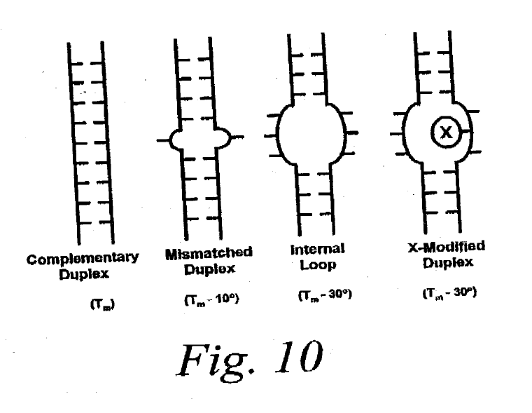

As shown in FIG. 10, when an oligonucleotide (15-20 bases) is hybridized with a complementary strand of DNA, a single base mismatch results in a thermal melting point (Tₘ) that is about 10° C. lower than the Tₘ of a perfectly complementary duplex. An internal loop of non-complementary sequences further destabilizes hybridization and it is expected that a chemical modification of even a single functionality will exhibit similar physical properties due to steric interference and/or conformational changes caused by the X moiety.

The reversibly modified nucleic acids are used to prevent or disrupt hybridization during routine sample preparation. Removal of the protecting groups after a short preincubation step will result in functional, single stranded nucleic acids that are made available for optimized hybridization stringency. Genomic methods require stringent hybridization conditions selected to be about 5° C. lower than the thermal melting point (Tₘ) for a specific sequence at a defined ionic strength and pH. However, a relaxed stringency of hybridization conditions occurs at room temperature during routine sample preparation steps resulting in mismatched hybrids, false readings or background noise. Thus, the invention provides for optimized hybridization stringency approached from a high temperature and fully denatured nucleic acids.

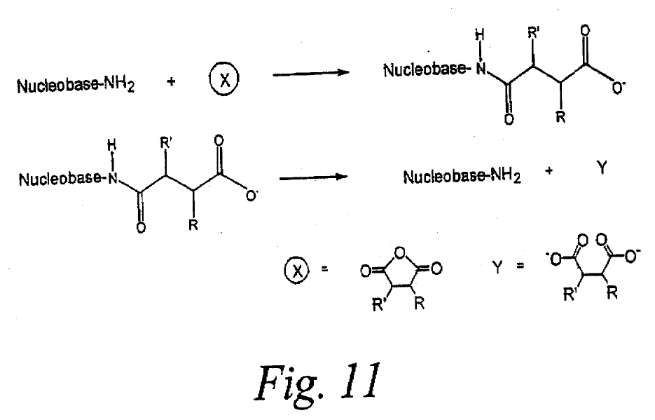

A library of modified nucleic acid model compounds that can be characterized for the conditions and kinetics of deprotection is considered to be within the scope of this invention. The coupling step involves reaction of the nucleobase amino groups with the protecting group (X): an anhydride, an imidoester, a carboxylic acid requiring some sort of activation (e.g. carbodiimide, acid chloride, etc.), or other reagent suitable for reaction at the nucleobase. A library of these compounds can be created by condensing deoxynucleosides and deoxynucleotide monophosphates of A, C, G or T with the reagents noted above. A representative reaction scheme is shown in FIG. 11, directed at nucleobase amino groups (e.g. N⁴ of cytidine, N⁶ of Adenine, N¹ or N² of Guanine, and N³ of Thymine) using a cyclic anhydride as the modifying group.

Genotyping by SNP (single nucleotide polymorphism) analysis and allele-specific oligonucleotide (ASO) hybridizations, which are the basis for microarray or DNA-Chip methods, are other genomic methods that are expected to benefit from a technology for enhanced accuracy of hybridization. Microarrays are constructed by arraying and linking PCR amplified cDNA clones or genes to a derivatized glass plate. Currently, the linking chemistries depend on high-salt buffers with formamide or dimethyl sulfoxide (DMSO) to denature the DNA and provide more single-stranded targets for eventual hybridization with high specificity and minimal background. This is a critical step in the preparation of reproducible, high-fidelity microarrays which may benefit from reversibly modified nucleic acids developed in this project. Further, the specific conditions of the pre-hybridization and hybridization steps can dramatically affect the signal from the microarray and technology from this project may be able to improve microarray performance at this step of the process.

Hypersensitive protecting groups may provide improved approaches to drug delivery. Controlled release of nucleic acid based pharmaceuticals such as AZT, dideoxy nucleic acids, or nucleic acid analogues may benefit from the technology. Also, for gene therapy, thermosensitive cationic polymers have been reported as non-viral DNA carriers for improved methods of transfection and efficient delivery of DNA to the nucleus of target cells. Thus, direct modification of nucleic acids with sensitive protecting groups developed in this project may provide interesting alternatives to consider for non-viral transfection in gene therapy. In addition, there may be applications of this chemistry to protein nucleic acid (PNA) technology. PNA probes are DNA mimics with unique properties and a growing number of applications have been developed for research applications and genomic methods. Lastly, the modified nucleic acids from this project are expected to provide nuclease resistance to nucleic acids which may have applications in therapeutics, i.e., these protecting groups may be used to increase in vivo survival times of antisense DNA.

The invention also provides kits for the convenient practice of the methods of the invention. In one embodiment, the invention provides a kit for performing PCR on a nucleic acid sample and, preferably, contains at least a reagent for reversibly chemically modifying a nucleic acid or nucleobase. The kit may also contain at least one other reagent for carrying out a polymerase reaction such as a modified primer capable of inhibiting the formation of undesired amplification products.

Diagnostic Applications

The methods, compositions, and kits of the invention are useful in a variety of diagnostic applications, such as the amplification and detection of nucleic acid sequences found in genomic DNA, bacterial DNA, fungal DNA, or viral RNA or DNA.

The methods and compositions described herein may also be used to detect or characterize nucleic acid sequences associated with infectious diseases, genetic disorders, or cellular disorders such as cancer.

The methods and compositions of the invention are also useful for the detection of certain types of non-genetic diseases. For example, many viruses function by incorporating their nucleic acid molecule into that of the host cell, in which it may lie dormant until a specific event triggers viral production. It is possible to use the methods and compositions of the invention (and appropriate primers known to one skilled in the art) to detect the presence of a viral nucleic acid molecule (e.g., HIV or hepatitis) within a nucleic acid sample derived from a human cell sample.

Similarly, the methods and compositions of the invention may be utilized to detect the presence of foreign cells in a subject. For example, the presence of microbes, e.g., bacteria, fungi, and/or viruses in various bodily fluids or tissues (e.g., blood, urine, or spinal fluid) can be detected using the improved polymerase amplification of the invention appropriately adapted to detect sequences specific to a microbial genome.

The methods and compositions of the invention may also be applied to the detection of cancerous cells, for example, by detecting specific chromosomal rearrangements (e.g., translocations) or changes in gene expression (e.g., by detecting the one or more selected mRNA molecules) in a nucleic acid sample derived from the cancer.

Forensic Applications

Forensic science is concerned with the scientific analysis of evidence from a crime. Forensic biology applies the experimental techniques of molecular biology, biochemistry, and genetics to the examination of biological evidence for the purpose, for example, of positively identifying the perpetrator of a crime. Typically, the sample size of such biological evidence (e.g. hair, skin, blood, saliva, or semen) is very small and often contains contaminating background DNA. Accordingly, the improved polymerase amplification techniques of the invention may be used to detect, e.g., the sex or species of origin of even minute biological samples.

Research Applications

The methods and compositions of the invention have a variety of research applications. For example, they are useful for any research application in which genetic analyses must be performed on limited amounts of a nucleic acid sample or in the presence of background DNA.

The following examples are included for purposes of illustration and should not be construed as limiting the invention.

### EXAMPLES

Throughout the examples, the following materials and methods were used unless otherwise stated.

Materials and Methods

In general, the practice of the present invention employs, unless otherwise indicated, conventional techniques of chemistry, molecular biology, recombinant DNA technology, PCR technology, immunology, and any necessary cell culture or animal husbandry techniques, which are within the skill of the art and are explained fully in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); Oligonucleotide Synthesis (M. J. Gait, Ed. 1984); PCR Handbook Current Protocols in Nucleic Acid Chemistry, Beaucage, Ed. John Wiley & Sons (1999) (Editor); Oxford Handbook of Nucleic Acid Structure, Neidle, Ed., Oxford Univ Press (1999); PCR Protocols: A Guide to Methods and Applications, Innis et al., Academic Press (1990); PCR Essential Techniques: Essential Techniques, Burke, Ed., John Wiley & Son Ltd (1996); The PCR Technique: RT-PCR, Siebert, Ed., Eaton Pub. Co. (1998); Quantitative PCR Protocols, Kochanowski et al., Eds., Humana Press (1999); Clinical Applications of PCR, Lo, Ed., Humana Press (1998); Current Protocols in Molecular Biology, eds. Ausubel et al., John Wiley & Sons (1992); Large-Scale Mammalian Cell Culture Technology, Lubiniecki, A., Ed., Marcel Dekker, Pub., (1990); and Manipulating the Mouse Embryo, Hogan et al., C.S.H.L. Press, Pub (1994).

PRIMER SET FOR AMPLIFYING PUC19

| | |
|---|---|
| Reverse Primer | |
| 5′ GCCGGATCAAGAGCTACCAAC 3′ | (SEQ ID NO: 1) |
| Forward Primer | |
| 5′ GGTTGTAAAACGACGGCCAGTG 3′ | (SEQ ID NO: 2) |

All primers were synthesized by standard phosphoramidite protocols (G. K. McMaster et al. (1977) PNAS USA, 74:4835; P. S. Thomas (1980) PNAS USA, 77:5201) and supplied by Sigma-Genosys, Houston, Tex., USA.

Primers were glyoxylated by preparing the following reaction mixtures which were allowed to stand at 55° C. in a sealed tube for 3 hr.

TABLE 1

| Component | Volume | Concentration |
|---|---|---|
| Primer-1 (P-1) | 200 | 25 mM |
| Glyoxal solution | 200 | 2% |
| tris-borate pH 8.3 | 40 | 10 x |
| Primer-2 (P-2) | 50 | 100 mM |
| Glyoxal solution | 50 | 2% |
| tris-borate pH 8.3 | 10 | 10 x |

Template DNA

Template DNA used was pUC-19 (New England Biolabs, Beverly Mass. USA).

PCR Reaction Mixes

Buffers, polymerases, mixes and other reaction components were obtained from Sigma except where otherwise stated.

Standard PCR reaction mixtures used contained the following with pUC19 as template DNA:

TABLE 2

| | |
|---|---|
| pUC19 | 10 ng |
| dNTPs | 200 mM in each |
| Forward primer | 500 nM, 50 pm |
| Reverse primer | 500 nM, 50 pm |
| MgCl2 | 2.5 mM |
| Buffer | (tris-borate, EDTA) 1 x (obtained from |
| | Sigma) |
| Taq DNA polymerase | 1.25 units (obtained from Sigma) |
| ddH₂O | to a total volume of 100 μl. |

Thermal Cycling Conditions

Unless otherwise indicated, the standard thermal cycling conditions used were:

TABLE 3

| | |
|---|---|
| 95° C. | 2 mins |
| 95° C. | 45 sec |
| 55° C. | 30 sec repeated for 35 cycles |
| 72° C. | 30 sec |
| 72° C. | 2 mins |

When the samples were pre-heated they were held at 95° C. for 10 minutes prior to using the protocol above.

Reactions were overlaid with 50 μl of mineral oil to prevent evaporation of reaction components during thermal cycling. Amplification was carried out using a Minicycler™ (Model No. PTC-150) from MJ Research (Watertown, Mass., USA).

Imaging

Unless otherwise indicated, nucleic acid products were resolved using 1.5% agarose gel (Sigma, St. Louis, Mo., USA) electrophoresis (conducted at approximately 70 mA for 2-3 hours) and visualized using ethidium bromide (Sigma) added directly to the gel solution at a 500 ng/ml and a Fisher Scientific Transilluminator (Model FBTIV-614). Gels were photographed using a Kodak DC120 Digital Zoom Camera.

### Example 1

### Reversibly Chemically Modified Nucleic Acid Amplification

In this example, methods for chemically modifying nucleic acids using glyoxyl and other analogues or derivatives are described. Additionally, the methods describe optimal amplification of nucleic acids using glyoxyl and other analogues or derivatives.

5′Dimethoxytrityl (DMT)-guanosine was prepared by allowing 12.5 mg of N6-isobutryl, 5′dimethoxytrityl guanosine (Raylo Chemicals, Edmonton, Alberta, Canada) to stand in 1 ml of 28% ammonium hydroxide (Fisher Chemical Co., Phila., Pa., USA) in a sealed tube at 55° C. for 12 hr. The product was isolated as an amorphous, white solid after removal of water and ammonia in a Speedvac concentrator (Savant Inc., Farmingdale, N.Y., USA) at 55° C. The crude product was dissolved in 1 ml of dimethylformamide-water (1:4).

Glyoxylated, 5′DMT-guanosine was prepared by mixing 50 μl of the crude, 5′DMT-guanosine solution (˜625 μg) with 50 μl of a 2% glyoxal:water solution prepared by diluting glyoxal (40% aqueous solution, Sigma Inc., St. Louis, Mo., USA) with water. The mixture was allowed to stand in a sealed tube at 55° C. for 2 hr. and the product was analyzed directly by HPLC.

Glyoxylated 5′DMT-guanosine was purified by preparative, reversed phase HPLC. A 50 μl aliquot of the crude, glyoxylated, 5′DMT-guanosine mixture was injected and component eluting between 10.5 to 11.5 min was collected and dried in the Speedvac at 55° C. The product was dissolved in 600 μl dimethylformamide:water (1:2) and this solution was used for all subsequent analysis and experimental work.

Glyoxylated, 5′DMT-guanosine was converted to DMT-guanosine by mixing equal volumes (50 μl) of the glyoxylated 5′DMT-guanosine solution and a buffer solution. The mixtures were allowed to stand at 55° C. in a sealed tube and aliquots were analyzed by HPLC using Gradient-1, defined below. Buffer solutions were (a) 0.10M sodium phosphate, pH 9.3, and (b) 10× Tris-Borate-EDTA Buffer (Sigma Inc., St. Louis, Mo.,USA), which is 0.89M Tris borate, pH approx. 8.3, containing 0.02M EDTA.

Samples were analyzed at 260 nm with a Waters (Milford, Mass., USA) 600 Solvent Delivery System and a Waters 486 variable wavelength UV detector. A 250×4.5 mm, C-18, Vydac column (Hesperia, Calif., USA, p/n 218TP54, 5 mm) was used with a gradient of solvent A (0.5 mM triethylammonium acetate, pH 7.0) to solvent B (acetonitrile). Gradient-1 was used for small molecule organics such as the DMT-guanosine model compounds: initial conditions at 75% A and 25% B and a linear gradient to 20% A and 80% B over 15 min. Gradient-2 was used for oligonucleotides: initial conditions at 98% A and 2% B and a linear gradient to 10% A and 90% B over 15 min. Injections were made by a Waters automatic sample injector (WISP™) or a Rheodyne manual injector.

EZChrom Elite software (Scientific Software, Inc., Pleasanton, Calif., USA) was used with the HPLC system for data handling and analysis. Integration of peaks was started at the 5 min. mark in the chromatograms to eliminate early solvent peaks from the area calculations.

Glyoxylated primers (described in Table 1) were purified by preparative, reversed phase HPLC using Gradient-2 with the Primer-1 mixture (final volume 400 ml) requiring two injections of 200 ml each. A total volume of approximately 1 ml was collected for the major component which eluted between 6.5 to 7.5 minutes. The samples were concentrated to dryness in a Speedvac at 55° C., redissolved in 200 ml water and frozen.

Concentrations of the glyoxylated primers were determined by comparison to the unmodified, primer stock solutions of known concentrations. The unmodified and glyoxylated primer solutions were analyzed by HPLC by Gradient-2 and the peak areas of these analyses were used to adjust the concentrations suitable for PCR reaction conditions. The HPLC peak areas for a 5 ml injection of the 200 ml solution of the glyoxylated primers (above) were used to determine necessary dilutions relative to the unmodified primer solutions according to the following data:

TABLE 4

| | | | | calc. peak | req. dilution |
|---|---|---|---|---|---|
| | | | | area for 5 ml | for a sol'n of |
| | | ml | | of a 10 mM | 5 ml @ |
| Sample | conc | injected | peak area | sol'n | 10 mM |
| P-1 | 100 mM | 5 | 8,454 | 845 | |
| P-2 | 25 mM | 20 | 8,630 | 863 | |
| Pg-1 | | 5 of 200 | 2,716 | | 3.15 |
| Pg-2 | | 5 of 200 | 1,114 | | 1.32 |

Accordingly, Pg-1 required a 3.15 fold dilution (419.3 ml water added to remaining 195 ml stock) and Pg-2 required a 1.32 fold dilution (59.2 ml added to remaining 185 ml stock).

A PE Biosystems (Framingham, Mass., USA) Voyager was used for matrix assisted, laser desorption, time of flight mass spectral analysis (MALDI-TOF-MS). Aliquots (16 ml, about 200 picomoles.) of the 10 mM primer solutions were submitted for mass spectral analysis to an outside laboratory Typical requirements for this data were about 65 picomoles per analysis or about 430 ngm.

Results and Discussion

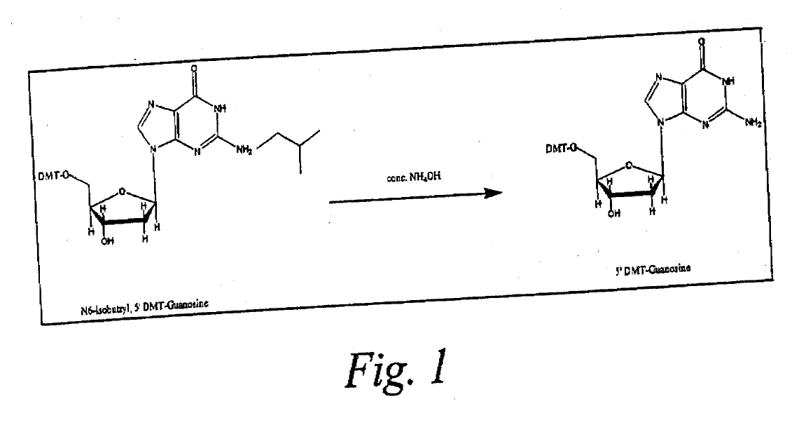

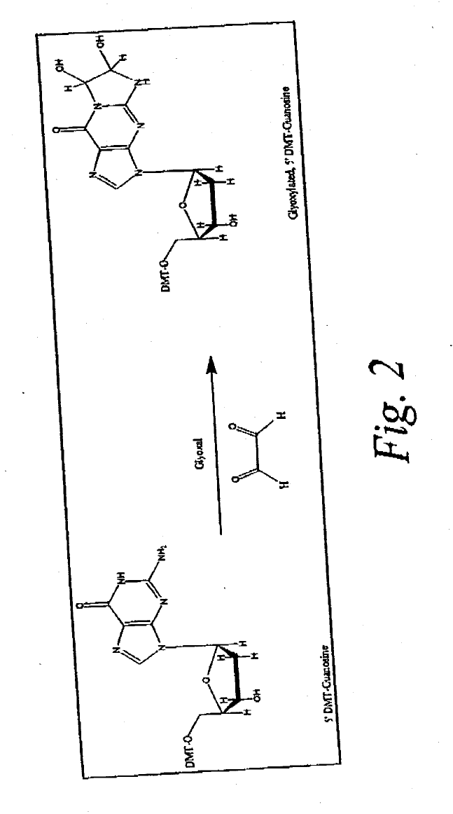

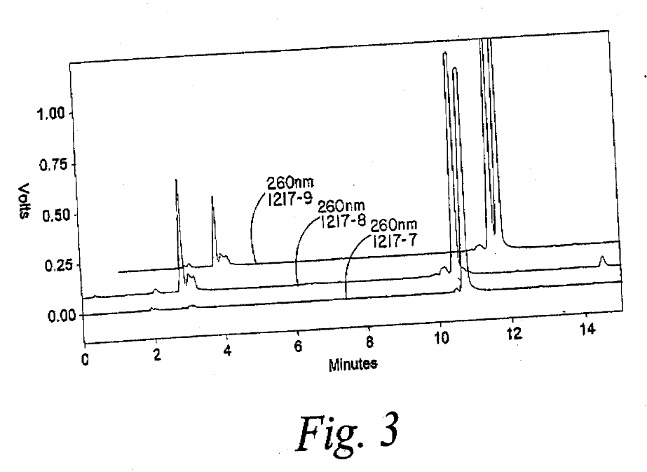

The model compound, 5′DMT-guanosine, was prepared from N6-isobutryl, 5′DMT-guanosine as shown in FIG. 1. The crude product was analyzed by HPLC and a single, major peak was observed. Glyoxylated, 5′DMT guanosine was prepared as shown in FIG. 2 and the product was characterized by HPLC. As shown in FIG. 3, co-injection of the starting material and the product establish that the compounds have distinct elution times with the glyoxylated product eluting slightly ahead of the starting material. The HPLC analysis of the crude, glyoxylated, 5′DMT-guanosine indicates a number of minor, unknown components which elute at the front and at about 14.5 minutes. These unknowns are most likely from glyoxal, glyoxal decomposition products, or decomposition of the DMT-guanosine resulting in guanine and dimethoxytrityl alcohol.

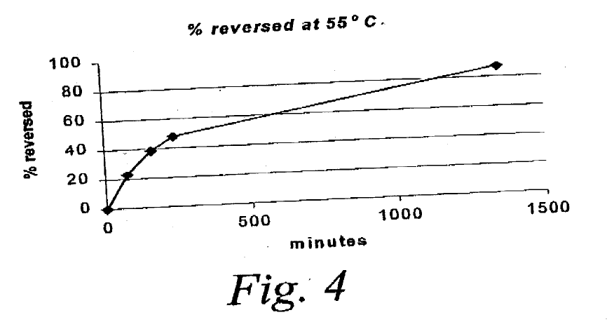

Glyoxylated, 5′DMT-guanosine was purified by preparative HPLC and tested for regeneration of 5′DMT-guanosine by treatment with (a) sodium phosphate buffer pH 9.3 and (b) tris-borate buffer pH 8.3. Equal amounts of the purified, glyoxylated 5′DMT-guanosine solution and the buffer solution were mixed and incubated at 55° C. in a sealed tube. After 30 minutes, an aliquot of the pH 9.3 mixture was analyzed by HPLC and it was found that 82.1% of the sample had been converted to the peak corresponding to 5′DMT-guanosine i.e. loss of the glyoxal group. The pH 8.3 mixture was analyzed at various time points over a 22 hour period and the data is presented in FIG. 4. This sample was 48.2% converted after 4 hours and 87.2% converted after 22 hours.

Forward and reverse primers for a 1005 base pair amplicon of pUC-19 were applied to the glyoxal reaction. A 25 mM aqueous solution of Primer-1, a 21 mer with 5 guanosine residues, and a 100 mM aqueous solution of Primer-2, a 22 mer with 8 guanosine residues were used directly. Reaction mixtures were prepared by dissolving the synthetic primer in the appropriate amount of water, adding an equal volume of 2% glyoxal in water, and adjusting the pH by the addition of 10× tris-borate, pH 8.3. The final conditions for the reaction were 1% glyoxal in 1× (0.089M) tris-borate buffer, pH 8.3.

Analysis of the oligonucleotides by HPLC showed no discernible differences for the elution times of the primer and the glyoxylated primer. Since reversed phase HPLC is unable to resolve minor changes in the hydrophobic or hydrophilic character of oligonucleotides. Indeed, oligonucleotides of any size and sequence from 3-30 residues typically elute within a narrow range of solvent conditions on a reversed phase column.

The glyoxylated primers were purified by preparative, reversed phase HPLC and the major component eluting between 6.5 to 7.5 minutes was isolated. Quantitative HPLC analysis of the primers was used to prepare 10 mM stock solutions suitable for application to PCR. The HPLC peak areas of the unmodified primer solutions with known molar concentrations were used as standards to determine necessary dilutions of the glyoxylated primer solutions such that primer concentrations could be adjusted to 10 mM. Thus, for amplification reactions, the volumes and concentrations of primers were consistent at the established 5 μl per 10 mM primer solution per PCR reaction.

The unmodified and glyoxylated primers were analyzed by MALDI-TOF-MS. As shown in Table 5, the theoretical molecular weight increase for primer-1 is 464 atomic mass units (amu) and for primer-2, 290 amu. The theoretical and measured Mᵣ for the primers is presented in the table and the measured values are in good agreement with predicted Mᵣ values. For both primers, the measured Mᵣ values for the unmodified primers and the measured Mᵣ values for the glyoxylated primers are within 1 amu of theoretical for addition of the glyoxyl moiety to only the guanosine residues in the oligonucleotide (58 amu per residue).

TABLE 5

| | | | | | Theo. | Measured |
|---|---|---|---|---|---|---|
| | Theo. | Measured | Total | Total G | (Mᵣ) | (Mᵣ) |
| Primer | (Mᵣ) | (Mᵣ) | Residues | Residues | Increase | Increase |
| P-1 | 6824 | 6826 | 22 | 8 | 464 | |
| P-2 | 6409 | 6416 | 21 | 5 | 290 | |
| Pg-1 | 7288 | 7291 | 22 | 8 | | 465 |
| Pg-2 | 6699 | 6705 | 21 | 5 | | 289 |

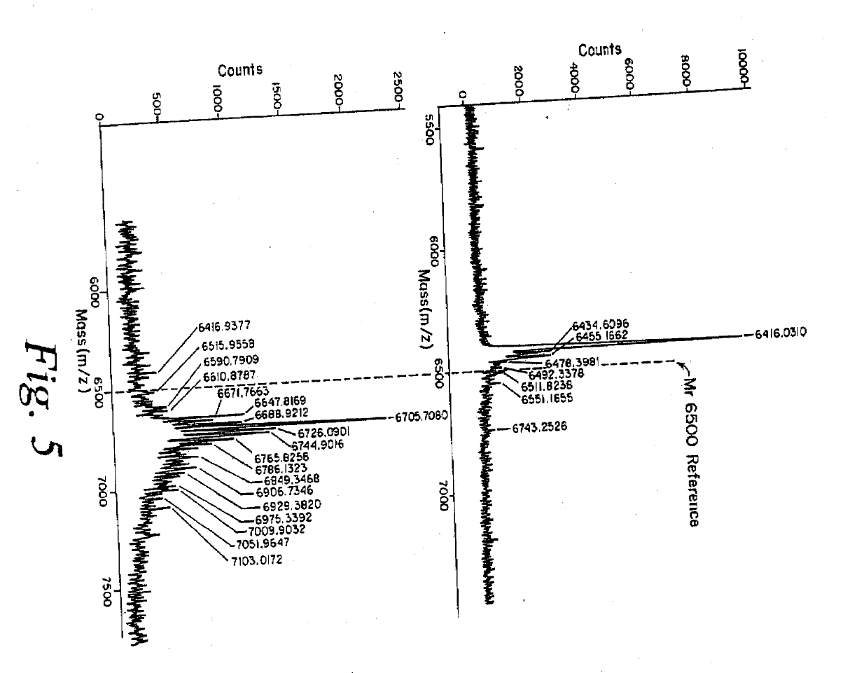

FIG. 5 presents the MALDI-TOF-MS data for the P-2 primer and the modified Pg-2 primer. The unmodified primer was an unpurified synthesis product and the spectrum for this sample indicates a major component with the expected molecular weight plus a number of other contaminants of higher molecular weight which may be from incomplete removal of protecting groups in the final step of the synthesis protocol. The spectrum for the glyoxylated primer indicates the major component with the expected increase higher mass. There are identified peaks at 6647 and 6590 amu which are attributable to the addition of 4 and 3 glyoxal moieties. A small peak is identified at 6416 amu which is the unmodified starting material. A close examination of peaks of higher mass indicates that they are attributable to the addition of 290 amu (5 glyoxal moieties) to the higher molecular weight components observed in the unmodified primer spectrum.

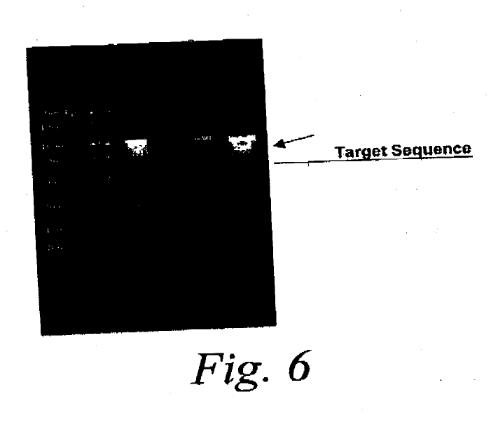

A 1005 bp fragment of pUC-19 was amplified by the PCR protocol and the data is presented in FIG. 6. Comparison of unmodified primers and glyoxylated primers with and without preincubation to remove the glyoxal protecting group is shown. Lane 1 is DNA size markers as indicated. The band corresponding to the amplified target sequence is indicated. As shown in lanes 2 and 5, amplifications using unmodified primers (P-1 and P-2) resulted in predominantly the target amplification product with non-specific amplification products noticeable above and below the target sequence. Comparison of lanes 2 and 5 demonstrates that preincubation has little or no effect on the amplification process with unmodified primers since lane 2 is the result of amplification without a preincubation step and lane 5 is the result with preincubation.

As shown in lane 3, use of the chemically modified, glyoxylated primers without preincubation in the PCR protocol, resulted in a significant decrease in the intensity of the band corresponding to the amplified target sequence, and a significant decrease in the intensity of the bands corresponding to the non-specific amplification products. In fact, for lane 3, the target sequence is obtained at only a very low level in comparison to lane 2 and 5 with the unmodified primers.

As shown in lane 4, use of the chemically modified, glyoxylated primers with preincubation in the PCR protocol, results in a strong signal for the band corresponding to the amplified target sequence, and decreased intensity of the bands corresponding to the non-specific amplification products compared to the results with unmodified primers (lanes 2 and 5). In addition, comparison of lane 4 to lane 3 confirms that the preincubation step in the PCR amplification process is effective in regenerating the unmodified primers and removing the glyoxyl blocking function with concomitant increase in the band intensity for the target sequence.

Comparison of lanes 4 and 5 indicate an overall reduction in signal using the glyoxylated primers even with preincubation. However, change in the relative amount of products within each lane best indicates the effect of pre-reaction treatments on non-specific amplification. Because the amplification of non-specific products competes with the amplification of the target sequence, an increase in amplification of the target sequence further indicates the amount of the reduction in non-specific amplification. This data indicate that PCR amplification using reversibly inactivated primers can significantly reduce non-specific amplification and can significantly increase the amount of desired amplified target sequence.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A method for amplifying a target nucleic acid comprising combining a target nucleic acid, under conditions which allow for an amplification reaction to occur, with:
a) one or more nucleic acid primers capable of binding to said target nucleic acid;
b) a nucleic acid polymerase; and
c) a plurality of nucleoside triphosphates,
wherein said target nucleic acid, said one or more primers or said plurality of nucleoside triphosphates have been reversibly modified so as to inhibit the formation of undesired amplification products, thereby forming a resultant mixture resulting in the selective amplification of the target nucleic acid.

2. The method of claim 1, wherein said reversibly modified target nucleic acid or said one or more primers comprise at least one nucleobase that has been reversibly modified chemically.

3. The method of claim 1, wherein said one or more primers have been reversibly modified so as to inhibit the formation of undesired amplification products.

4. The method of claim 1, wherein said amplification results in a reduced amount of non-specific nucleic acid amplification products.

5. The method of claim 2, wherein said reversibly modified nucleobase comprises a removable protecting group.

6. The method of claim 1, wherein said amplification is the PCR.

7. The method of claim 6, wherein said amplification is hot-start PCR.

8. The method of claim 5, wherein said removable protecting group is selected from the group consisting of glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal.

9. The method of claim 5, wherein said removable protecting group is glyoxal.

10. The method of claim 5, wherein said removable protecting group is selected from the group consisting of 3,4,5,6-tetrahydrophthalic anhydride, 3-ethoxy-2-ketobutyraldehyde(kethoxal), ninhydrin, hydroxyacetone, diethyl oxalate, diethyl mesoxalate, 1,2-naphthoquinone-4-sulfonic acid, and pyruvaldehyde.

11. The method of claim 5, wherein said removable protecting group is selected from the group consisting of amides; γ-Carboxyacylamides; amidines; and carbamates.

12. The method of claim 11, wherein said amide is selected from the group consisting of trifluoroacetyl, and trichloroacetyl.

13. The method of claim 11, wherein said γ-Carboxyacylamide is selected from the group consisting of acontinoyl, maleyl, citriconyl, phenoxyacetyl, and acetoacetyl.

14. The method of claim 11, wherein said amidine is an imidoamide.

15. The method of claim 11, wherein said carbamate is an ethoxycarbonyl.

16. The method of claim 1, wherein said resultant mixture is heated.

17. The method of claim 1, wherein said resultant mixture is heated for a period of time sufficient to remove said protecting group.

18. The method of claim 1, wherein said polymerase is selected from the group consisting of E. coli DNA polymerase I, TAQ polymerase, Klenow fragment of E. coli DNA polymerase I, T4 DNA polymerase, reverse transcriptase, and thermostable DNA polymerase.

19. The method of claim 1, wherein said polymerase is a thermostable DNA polymerase.

20. The method of claim 1, wherein said resultant reaction mixture is subjected to at least one thermal cycle.

21. An amplified nucleic acid produced by a process comprising combining a target nucleic acid, under conditions which allow for an amplification reaction to occur, with:
a) one or more nucleic acid primers capable of binding to said target nucleic acid;
b) a nucleic acid polymerase; and
c) a plurality of nucleoside triphosphates,
wherein said target nucleic acid, said one or more primers or said plurality of nucleoside triphosphates have been reversibly modified so as to inhibit the formation of undesired amplification products, thereby forming a resultant mixture resulting in the selective amplification of the target nucleic acid.

22. The amplified nucleic acid of claim 21, wherein said polymerase is a thermostable DNA polymerase.

23. The amplified nucleic acid of claim 22, wherein said resultant reaction mixture is subjected to at least one thermal cycle.

24. A kit for conducting a polymerase amplification reaction comprising a reagent for reversibly chemically modifying a nucleic acid or nucleobase such that when said nucleic acid is used in a polymerase amplification reaction the formation of undesired amplification products is inhibited; and instructions for use.

25. The kit of claim 24, wherein said kit further comprises a component selected from the group consisting of a nucleic acid primer and a nucleic acid polymerase.

26. The kit of claim 24, wherein said kit further comprises at least one other component for conducting a polymerase amplification reaction.

27. The kit of claim 24, wherein said kit comprises a thermostable DNA polymerase.

28. The kit of claim 24, wherein said reagent comprises a buffer and a removable protecting group.

29. The kit of claim 28, wherein said removable protecting group comprises glyoxal or glyoxal analogues or derivatives.

30. The kit of claim 28, wherein said removable protecting group is selected from the group consisting of glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal.

31. The kit of claim 24, wherein said removable protecting group is selected from the group consisting of glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal.

32. The kit of claim 24, wherein said removable protecting group is glyoxal.

33. The kit of claim 24, wherein said removable protecting group is selected from the group consisting of 3,4,5,6-tetrahydrophthalic anhydride, 3-ethoxy-2-ketobutyraldehyde(kethoxal), ninhydrin, hydroxyacetone, diethyl oxalate, diethyl mesoxalate, 1,2-naphthoquinone-4-sulfonic acid, and pyruvaldehyde.

34. The kit of claim 24, wherein said removable protecting group is selected from the group consisting of amides; γ-Carboxyacylamides; amidines; and carbamates.

35. The kit of claim 24, wherein said amide is selected from the group consisting of trifluoroacetyl, and trichloroacetyl.

36. The kit of claim 24, wherein said γ-Carboxyacylamide is selected from the group consisting of acontinoyl, maleyl, citriconyl, phenoxyacetyl, and acetoacetyl.

37. The kit of claim 24, wherein said amidine is an imidoamide.

38. The kit of claim 24, wherein said carbamate is an ethoxycarbonyl.

39. A compound having the capability of amplifying a target nucleic acid and reducing undesired amplification products, comprising a reaction mixture of a removable protecting group and guanosine 5′-triphosphate.

40. The compound of claim 39 wherein said removable protecting group has the following structure: 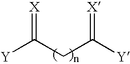
wherein n=0 to 4; Y and Y′ are selected from the group consisting of Cl, OH, H, NR₂, and OR; wherein R is an alkyl group selected from the group consisting of methyl, ethyl and isopropyl; and wherein X and X′ are selected from the group consisting of O and S.

41. The compound of claim 40 wherein said removable protecting group is selected from the group consisting of glyoxal dihydrate, glyoxal hydrogen sulfite, 2,3-dihydroxy-1,4-dioxane, pyruvaldehyde(methylglyoxal), 2,3-butanedione(dimethylglyoxal), 2,3-pentanedione(ethylmethylglyoxal), phenylglyoxal, and di-3-pyridylgloxal.

42. The compound of claim 40 wherein said removable protecting group is selected from the group consisting of 3,4,5,6-tetrahydrophthalic anhydride, 3-ethoxy-2-ketobutyraldehyde(kethoxal), ninhydrin, hydroxyacetone, diethyl oxalate, diethyl mesoxalate, 1,2-naphthoquinone-4-sulfonic acid, and pyruvaldehyde.
